(19) United States
(12) Patent Application Publication
(10) Pub. No.: US 2026/0056094 A1
(43) Pub. Date: Feb. 26, 2026

(54) ANALYSIS DEVICE

(71) Applicant: KYOCERA CORPORATION, Kyoto-shi, Kyoto (JP)

(72) Inventors: Shinichi ABE, Kyoto-shi, Kyoto (JP); Daisuke UEYAMA, Kyoto-shi, Kyoto (JP); Emiko NAKAGAWA, Kyoto-shi, Kyoto (JP)

(21) Appl. No.: 18/851,008

(22) PCT Filed: Mar. 29, 2023
(86) PCT No.: PCT/JP2023/012781

§ 371 Date: May 22, 2025

(30) Foreign Application Priority Data
Mar. 29, 2022 (JP) 2022-053887

(51) Int. Cl. G01N 1/24; E03D 9/00; G01N 33/483

(52) U.S. Cl. CPC G01N 1/24; E03D 9/00; G01N 33/4833

(57) ABSTRACT

To provide an analysis device capable of smoothly executing analysis of collected gas. An analysis device includes a reservoir (151) made of a flexible material for storing a sample gas collected through a first flow channel (31), an analysis chamber (18) for analyzing the sample gas supplied from the reservoir (151), and a second flow channel (32) for connecting the reservoir (151) and the analysis chamber (18), wherein a volume of the analysis chamber (18) is smaller than a volume of the reservoir (151), and the volume of the reservoir (151) is greater than or equal to the sum of the volume of the analysis chamber (18) and the volume of the second flow channel (32).

### TECHNICAL FIELD

The present disclosure relates to analysis devices, and more particularly to an analysis device for analyzing gas discharged from a user's body.

### BACKGROUND OF INVENTION

A system that detects an odorous gas generated from feces discharged by a user is known. For example, Patent Document 1 discloses a biological information measurement system that can be easily purchased by general consumers, and helps prevent diseases by measuring defecation gas at home.

### CITATION LIST

### Patent Literature

Patent Document 1: JP 2016-145809 A

### SUMMARY

An analysis device according to one aspect of the present disclosure includes a reservoir that is made of a material having flexibility and that stores sample gas collected through a first flow channel from a target; an analysis chamber for analyzing the sample gas supplied from the reservoir; and a second flow channel connecting the reservoir and the analysis chamber; where a volume of the analysis chamber is smaller than a volume of the reservoir, and a volume of the reservoir is greater than or equal to a sum of a volume of the analysis chamber and a volume of the second flow channel.

### BRIEF DESCRIPTION OF THE DRAWINGS

FIG. 1 is an example of an external view illustrating a configuration of an analysis system according to a first embodiment of the present disclosure.

FIG. 2 is an external view illustrating a state in which a part of the configuration of the analysis system illustrated in FIG. 1 is viewed from another viewpoint.

FIG. 3 is a block diagram illustrating an example of a configuration of a main part of the analysis device illustrated in FIG. 1.

FIG. 4 is a view illustrating an example of an appearance of the analysis device illustrated in FIG. 1.

FIG. 5 is a piping diagram of the analysis device illustrated in FIG. 1.

FIG. 6 is a view illustrating a configuration example of a first reservoir.

FIG. 7 is a diagram illustrating a configuration example of an analysis unit.

FIG. 8 is a flowchart illustrating an example of a flow of processes executed by the control unit of the analysis device illustrated in FIG. 3.

FIG. 9 is a part of a piping diagram of an analysis device according to a second embodiment of the present disclosure.

FIG. 10 is a part of a piping diagram of an analysis device according to a third embodiment of the present disclosure.

FIG. 11 is a part of a piping diagram of an analysis device according to a fourth embodiment of the present disclosure.

### DESCRIPTION OF EMBODIMENTS

Hereinafter, a first embodiment of the present disclosure will be described with reference to the drawings. Hereinafter, an analysis system 100 that detects gas generated from feces discharged by a user will be described as an example. The present disclosure is not limited to this example, and may be applied to any analysis device that detects gas generated in a predetermined space, for example, an analysis system that detects gas generated in a space such as a warehouse or a laboratory.

### First Embodiment

### Configuration of Analysis System 100

Hereinafter, a configuration of an analysis system 100 according to a first embodiment of the present disclosure will be described in detail with reference to the drawings.

Each of the figures referred to in the present specification is a schematic diagram illustrating only some members in a simplified manner in order to describe the embodiment for the sake of convenience of description. Thus, the analysis system 100 may include any constituent members not illustrated in the drawings to which the present specification refers. The dimensions of the members in the drawings do not faithfully represent the actual dimensions of the constituent members, the dimension ratios of the members, or the like.

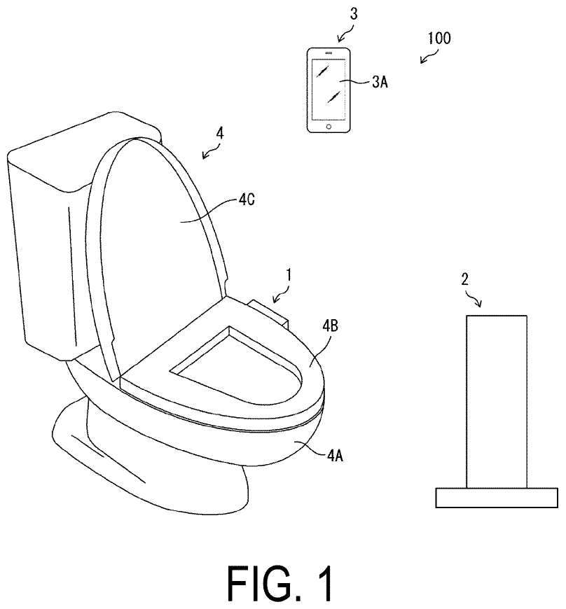

FIG. 1 is an example of an external view illustrating a configuration of an analysis system 100 according to a first embodiment of the present disclosure. As illustrated in FIG. 1, the analysis system 100 includes an analysis device 1, a server device 2, and a terminal device 3.

The analysis device 1 is installed in a toilet 4 including a toilet bowl 4A, a toilet seat 4B, and a lid 4C, and acquires gas generated from feces of a user discharged into the toilet bowl 4A as sample gas. Examples of the gas generated from the feces of the user include gases such as carbon dioxide, hydrogen, and methane, and/or odorous gases such as methyl mercaptan and hydrogen sulfide. The analysis device 1 detects the type of gas, the concentration of gas, and the like contained in the acquired sample gas by a sensor unit 18 described later.

The analysis device 1 may be installed, for example, near a side portion of the toilet bowl 4A or the toilet seat 4B. A part of the analysis device 1 may be embedded in the toilet bowl 4A or the toilet seat 4B. The toilet 4 may be a flush toilet, but is not limited thereto. The toilet 4 may be installed in a toilet room of a house, a hospital, or the like.

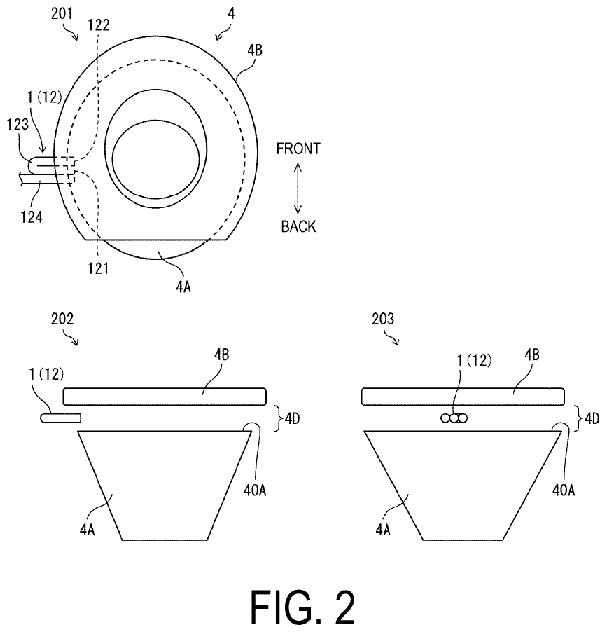

FIG. 2 is an external view illustrating a state in which a part of the configuration of the analysis system 100 illustrated in FIG. 1 is viewed from another viewpoint. A view indicated by reference numeral 201 in FIG. 2 is a schematic view illustrating a state in which the toilet 4 is viewed from above. Here, a direction indicated by an arrow described as front is referred to as front side, and a direction indicated by an arrow described as back is referred to as back side. A direction orthogonal to these arrows and substantially parallel to the plane of drawing is referred to as a side.

The drawing denoted by reference numeral 202 and the drawing denoted by reference numeral 203 in FIG. 2 are schematic views illustrating a state in which the toilet 4 is viewed from the back side and the side, respectively. In the views in FIG. 2, a configuration of the toilet 4 is partially omitted and exaggerated.

As illustrated in FIG. 2, the toilet bowl 4A includes an upper surface 40A on a side facing the toilet seat 4B. The toilet seat 4B may include, for example, four cushions (not illustrated) on a surface facing the upper surface 40A. When the toilet seat 4B is placed on the toilet bowl 4A, a gap 4D may be formed between the upper surface 40A of the toilet bowl 4A and the toilet seat 4B due to abutment between the cushion and the upper surface 40A. In the gap 4D, the user may arrange a suction tube 124, an intake portion 121, an exhaust portion 122, a pipe portion 123, and the like of an airflow generation unit 12, which will be described later.

This will be described again with reference to FIG. 1. The server device 2 is communicably connected to the analysis device 1 and a terminal device 3, and can receive information indicating an analysis result by the analysis device 1 from the analysis device 1 by wireless communication or wired communication. The server device 2 can estimate the health condition of the user based on the analysis result by the analysis device 1 and transmit information indicating the estimated health condition to the terminal device 3.

In the above description, an example in which the server device 2 estimates the health condition of the user has been described, but the configuration is not limited thereto. For example, the analysis device 1 may further include some of the functions of the server device 2, and the analysis device 1 may estimate the health condition of the user. The analysis device 1 can transmit an estimation result to the server device 2.

The estimation method may be, for example, a learned artificial intelligence (AI) or the like capable of estimating the health condition of the user based on the type of gas, the concentration of the gas, and the like contained in the sample gas.

The terminal device 3 is realized by, for example, a smartphone or the like used by the user. However, the terminal device 3 is not limited to a smartphone, and may be any electronic device such as a tablet. When brought into the toilet room by the user, the terminal device 3 exists inside the toilet room as illustrated in FIG. 1, but when not brought into the toilet room by the user, the terminal device 3 may exist outside the toilet room.

The terminal device 3 can receive information indicating the health condition of the user from the server device 2 by wireless communication or wired communication. The terminal device 3 may present the health information of the user to the user by displaying the received information on the display unit 3A. The terminal device 3 may notify the user of the health information of the user by voice through a speaker provided in the terminal device 3.

Thus, the user can easily acquire the information indicating the health condition. For example, the user can easily acquire the information indicating the health condition in a toilet room at home or the like without going to a hospital or the like.

The display unit 3A may include a display capable of displaying characters and the like, and a touch screen capable of detecting contact of a user's finger or the like. This display may include a display device such as a liquid crystal display (LCD), an organic electro-luminescence display (OELD), or an inorganic electro-luminescence display (IELD). A detection method of the touch screen may be any method such as a capacitive method, a resistive film method, a surface acoustic wave method (or an ultrasonic method), an infrared method, an electromagnetic inductive method, or a load detection method.

### Analysis Device 1

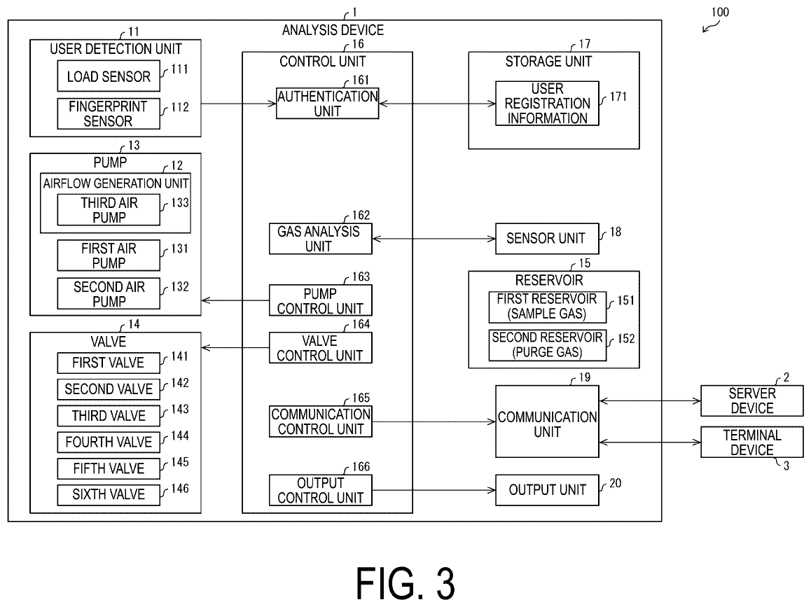

A configuration of the analysis device 1 of the first embodiment will be described with reference to FIG. 3. FIG. 3 is a block diagram illustrating an example of a configuration of a main part of the analysis device 1 illustrated in FIG. 1.

As described above, the analysis device 1 is installed in the toilet bowl 4A, acquires sample gas containing gas generated from feces of a user, and detects and analyzes the type of gas, the concentration of gas, and the like contained in the acquired sample gas. The analysis device 1 can transmit information indicating the result of the analysis to the server device 2.

As illustrated in FIG. 3, the analysis device 1 includes a user detection unit 11, an airflow generation unit 12, a pump 13, a valve 14, a reservoir 15, a control unit 16, a storage unit 17, a sensor unit 18 which is an analysis chamber, a communication unit 19, and an output unit 20.

### User Detection Unit 11

The user detection unit 11 includes an arbitrary sensor for detecting a user. As an example, the user detection unit 11 is configured including at least one of an infrared sensor, a pressure sensor, an image camera, and the like. The user detection unit 11 may be, for example, a sensor capable of detecting a user by recognizing a mobile terminal or the like owned by the user and associated with the user.

When the user detection unit 11 is configured including, for example, an infrared sensor, the user detection unit 11 detects that the user has entered the toilet room by detecting reflected light from an object of the infrared ray emitted by the infrared sensor. When detecting that a user has entered the toilet room, the user detection unit 11 outputs a signal indicating that the user has entered the toilet room to the control unit 16.

When the user detection unit 11 is configured including, for example, a pressure sensor, the user detection unit 11 detects pressure applied to the pressure sensor installed on the toilet seat 4B, thereby detecting that the user has sat on the toilet seat 4B. When detecting that the user has sat on the toilet seat 4B, the user detection unit 11 outputs a signal indicating that the user has sat on the toilet seat 4B to the control unit 16.

The pressure sensor detects that the user has stood from the toilet seat 4B by detecting a decrease in pressure applied to the toilet seat 4B. When the user detection unit 11 detects that the user has stood up from the toilet seat 4B, the user detection unit 11 outputs a signal indicating that the user has stood up from the toilet seat 4B to the control unit 16.

The user detection unit 11 may include a sensor that acquires data indicating physical characteristics in order to specify the user after detecting the user. The user detection unit 11 may include only a sensor that specifies the user by operation of the user without including a sensor that detects the user.

The user detection unit 11 includes, for example, at least one of a load sensor 111 and a fingerprint sensor 112. With either one of these sensors, the user detection unit 11 acquires information that can specify the user, and transmits the information to an authentication unit 161. The authentication unit 161 can authenticate the user based on the received information. The user detection unit 11 may further include a sensor that detects a sitting height, a sensor that detects a face, a sensor that detects voice, and the like. By further including these sensors, the user detection unit 11 can acquire information for accurately specifying the user.

The analysis device 1 stores the sample gas in a first reservoir 151 after the user is authenticated by the authentication unit 161. For example, the analysis device 1 starts storing the sample gas in the first reservoir 151 after a predetermined time has elapsed from the time point at which the user is authenticated by the authentication unit 161. For example, the analysis device 1 may start storage of the sample gas in the first reservoir 151 by a pump 13, a valve 14, or the like, to be described later, after a predetermined time has elapsed from the time point at which the user is authenticated by the authentication unit 161. For example, the control unit 16 of the analysis device 1 may start storage of the sample gas in the first reservoir 151 by controlling the pump 13 or the valve 14, to be described later, after a predetermined time has elapsed from the time point at which the user is authenticated by the authentication unit 161. Alternatively, the authentication of the user may be performed by the authentication unit 161 after the sample gas is stored in the first reservoir 151.

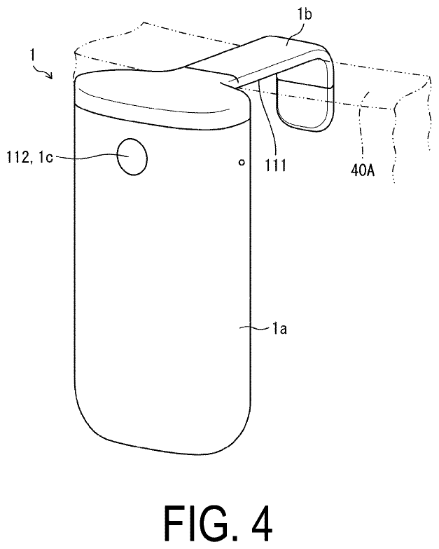

The authentication of the user by the analysis device 1 will be described in detail with reference to FIG. 4. FIG. 4 is a diagram illustrating an example of an external appearance of the analysis device 1.

As illustrated in FIG. 4, the analysis device 1 includes, for example, a main body la, an attachment portion 1b, an activation switch 1c, and a load sensor 111. The analysis device 1 may further include an airflow generation unit 12 (not illustrated).

The analysis device 1 may be disposed such that the attachment portion 1b is placed on the upper surface 40A of the toilet bowl 4A, and the attachment portion 1b is located between the upper surface 40A and the toilet seat 4B. The load sensor 111 is provided on the attachment portion 1b. For example, the load sensor 111 is located on the lower surface of the attachment portion 1b. When the user sits on the toilet seat 4B, the back surface of toilet seat 4B abuts on the upper surface of the load sensor 111 to receive a load, and the load sensor 111 can detect the weight of the user.

FIG. 4 illustrates a configuration example of the analysis device 1 in which the load sensor 111 and the main body la are integrated, but the configuration is not limited thereto. For example, the analysis device 1 may be a separate body from the load sensor 111. In this case, the user may install, for example, one analysis device 1 and a plurality of load sensors 111 for one toilet 4.

For example, the load sensor 111 may be located between the toilet bowl 4A and the toilet seat 4B. The accuracy of detecting the weight of the user can be improved by having the user arrange the plurality of load sensors 111 between the toilet bowl 4A and the toilet seat 4B. Therefore, even when there are a plurality of users of the toilet 4, if the weight of each user is different, the authentication unit 161 can authenticate each user based only on the weight detected by the load sensor 111. As a typical example of the case where the weight of each user is different, there is a case where a father, a mother, and a child use the toilet 4 installed in a house of a certain family, the weight of the father is heavier than the weight of the mother, and the weight of the child is lighter than the weight of the mother.

The activation switch 1c is a switch that activates the analysis device 1, and the user can activate the analysis device 1 by pressing the activation switch 1c with a finger. The activation switch 1c may be provided with a proximity sensor, and the user may activate the analysis device 1 by simply bringing a finger close to the activation switch 1c without touching the activation switch 1c. Thus, the user can activate the analysis device 1 hygienically without directly touching the activation switch 1c.

This is not the sole case, and for example, the analysis device 1 may be configured to be activated in conjunction with the load sensor 111 when the user sits on the toilet seat 4B. For example, the analysis device 1 may be configured to be activated when the user detection unit 11 detects a user.

The activation switch 1c also serves as the fingerprint sensor 112 that detects a fingerprint, and the activation switch 1c may be configured to acquire “fingerprint information” when the user brings the finger into contact with or close to the activation switch 1c.

An example of a procedure for authenticating a user by the analysis device 1 will be described in detail. Hereinafter, a case where the user detection unit 11 includes the load sensor 111 and the fingerprint sensor 112 will be described as an example. However, the authentication is not limited thereto, and the analysis device 1 can use, for example, a face authentication method using a face image, authentication using a sitting height, or body electric resistance.

The load sensor 111 detects a load applied to the toilet seat 4B when the user sits on the toilet seat 4B, and notifies the authentication unit 161 of the detected load as weight information of the user. The authentication unit 161 compares the weight information notified from the load sensor 111 with the weight information stored in the user registration information 171, extracts the user information including the matching weight information, and authenticates the user.

The user registration information 171 is, for example, a list in which weight information, fingerprint information, and user information are associated with each other. Here, the weight information is information of a numerical value of the weight of the user, and the fingerprint information is information such as an image for specifying the fingerprint of the user. The user information is information for specifying each user, and may be information including a name, an age, a gender, a user ID, an account, an e-mail address, and the like.

An application that acquires information generated by the above-described analysis device 1 and manages the health condition of the user may be created. The user can create an account for specifying the user in the application and log in with the account to display the information regarding the health condition of the user on the display unit 3A of the terminal device 3 of the user.

The authentication unit 161 acquires the account of the user from the extracted user information. The control unit 16 transmits the information regarding the health condition of the user to the server of the application via the communication control unit 165 so that the information regarding the health condition of the user can be displayed in the application.

The authentication unit 161 acquires the name, the e-mail address, the user ID, or the like of the user from the extracted user information. As a result, the analysis device 1 can transmit information indicating the type of gas, the concentration of gas, and the like contained in the sample gas of the user to the server device 2 together with information such as the name, the e-mail address, the user ID or the like of the user. When the authentication unit 161 acquires the e-mail address of the user, the server device 2 generates information indicating the health condition of the user that can be estimated from the received information on the sample gas, and transmits the information to the received e-mail address. Alternatively, this is not the sole case, and the information indicating the health condition of the user may be generated by the analysis device 1, and the analysis device 1 may transmit the information indicating the health condition of the user to the server device 2.

At this time, for example, when the weight information matching the weight information detected by the load sensor 111 is not stored in the user registration information 171 because the weight of the user has changed, the analysis device 1 cannot perform authentication of the user based on the weight information. In this case, for example, by sounding or blinking an alarm or an LED realized as the output unit 20 to be described later, the analysis device 1 may notify the user that the authentication of the user based on the weight information cannot be performed. Note that when the weight information that matches the weight information detected by the load sensor 111 is not stored in the user registration information 171, for example, the authentication unit 161 may estimate that the user associated with the weight information closest to the weight information detected by the load sensor 111 is using the analysis device.

When the authentication of the user based on the weight information cannot be performed, the user detection unit 11 may authenticate the user based on fingerprint information instead of the authentication based on weight information. As described above, the fingerprint sensor 112 also serves as the activation switch 1c, and can acquire fingerprint information when the user brings the finger into contact with or close to the activation switch 1c. When the authentication of the user based on the weight information cannot be performed, the user detection unit 11 may prompt the user to authenticate the user by the fingerprint information.

Accordingly, when the authentication of the user based on the weight information cannot be performed, the user detection unit 11 acquires the fingerprint information in accordance with the operation of the activation switch 1c by the user, and transmits the fingerprint information to the authentication unit 161. Thus, the analysis device 1 can authenticate the user.

The activation switch 1c may be disposed at a place where a user just touches by hand when the user sits on toilet seat 4B. As a result, the analysis device 1 can easily perform fingerprint authentication without requiring the user to perform an extra operation. For example, the activation switch lc may be located on the upper surface or the side surface of the analysis device 1.

The authentication unit 161 may authenticate the user based on only the fingerprint information. The authentication unit 161 compares the fingerprint information detected by the fingerprint sensor 112 with the fingerprint information stored in the user registration information 171, extracts user information including matching fingerprint information, and authenticates the user. The authentication unit 161 acquires the name, the e-mail address, the user ID, or the like of the user from the extracted user information.

The authentication unit 161 may perform authentication based on the weight information together with the fingerprint information. The authentication unit 161 may automatically perform authentication based on the weight information when the user forgets to perform authentication based on fingerprint information. When the load sensor 111 detects the weight information before the fingerprint sensor 112 detects the fingerprint information, the authentication unit 161 may automatically perform authentication based on the weight information. When the fingerprint sensor 111 does not detect the fingerprint information within a predetermined time after the load sensor 112 detects the weight information, the authentication unit 161 may automatically perform authentication based on the weight information.

The analysis device 1 may include a sterilization device. The sterilization device may be provided, for example, at the periphery of the activation switch 1c. The sterilization device can, for example, sterilize the fingers of the user by irradiating the fingers of the user with ultraviolet rays having wavelengths of about 222 nm.

The fingerprint sensor 112 may not be provided on the activation switch 1c but may be provided on, for example, a door knob on the inner side or a door knob on the outer side of the door of the toilet room. As a result, when the user opens the door or closes the door after entering the room, the analysis device 1 can perform the fingerprint authentication together with the opening/closing operation of the door.

The fingerprint sensor 112 may be provided on a flush switch for flushing the toilet bowl 4A.

### Airflow Generation Unit 12

Here, the airflow generation unit 12 will be described with reference to FIG. 2 again. As illustrated in FIG. 2, the airflow generation unit 12 creates a circulating flow of gas (airflow of sample gas) containing a sample gas in the toilet 4. As described above, the airflow generation unit 12 is provided in the gap 4D between, for example, the toilet bowl 4A and the toilet seat 4B.

However, the airflow generation unit 12 is not limited thereto, and for example, at least one part may be embedded in the toilet 4. For example, the airflow generation unit 12 may be provided integrally with the toilet 4.

The airflow generation unit 12 may include an intake portion 121, an exhaust portion 122, a pipe portion 123, and a third air pump 133 (see FIG. 3).

The intake portion 121 takes in a sample gas in the toilet bowl 4A. The exhaust portion 122 exhausts the sample gas taken in by the intake portion 121 into the toilet bowl 4A. The intake portion 121 and the exhaust portion 122 are connected to each other by a pipe portion 123.

The third air pump 133 is connected to the pipe portion 123, causes the gas in the toilet bowl 4A to be taken in by the intake portion 121 and discharged from the exhaust portion 122 via the inside of the pipe portion 123. The third air pump 133 may be configured by a piezo pump, a motor pump, or the like.

The airflow generation unit 12 takes in air from the intake portion 121 and exhausts air from the exhaust portion 122, thereby generating an airflow in the toilet bowl 4A. After being generated from the feces of the user, the sample gas is accumulated at the bottom of the toilet bowl 4A, but is wound up by the airflow generated by the airflow generation unit 12, and flows to the upper side of the toilet bowl 4A (on the lid 4C side), particularly, toward a direction in which a suction tube 124 to be described later is provided. As a result, the analysis device 1 can more efficiently collect the sample gas from the suction tube 124.

The airflow generation unit 12 takes in gas in the toilet bowl 4A and exhausts the gas into the toilet bowl 4A. Therefore, the likelihood that air outside the toilet bowl 4A flows into the toilet bowl 4A and the likelihood that gas inside the toilet bowl 4A flows out of the toilet bowl 4A are reduced. Thus, the generation of the airflow by the airflow generation unit 12 reduces the likelihood that a concentration of the sample gas in the toilet bowl 4A decreases.

The sample gas accumulated at the bottom of the toilet bowl 4A can be wound up by the airflow generated by the airflow generation unit 12 by directing the distal end of the exhaust portion 122 toward the bottom direction of the toilet bowl 4A. Thus, the concentration of the sample gas around the suction tube 124 can be increased, whereby the analysis device 1 can measure the concentration of the gas contained in the sample gas with higher accuracy.

### Pump 13 and Valve 14

This will be described again with reference to FIG. 3. The analysis device 1 includes one or more pumps 13 capable of moving the sample gas along at least one of a first flow channel 31 and a second flow channel 32 described later. As will be described in detail later, the flow rate of the sample gas in the first flow channel 31 is controlled to be greater than or equal to the flow rate of the sample gas in the second flow channel 32.

The analysis device 1 includes a plurality of pumps 13 that supply or discharge sample gas or purge gas. Specifically, the analysis device 1 includes a first air pump (first pump) 131, a second air pump (second pump) 132, and a third air pump 133. Each of the plurality of pumps 13 may be configured by a piezo pump, a motor pump, or the like. In the following description, the first air pump 131, the second air pump 132, the third air pump 133, and the like are not distinguished from each other, and when they are described for the purpose of being collectively referred to, they are referred to as the pump 13.

The analysis device 1 also includes a plurality of valves 14 for switching the flow direction of the sample gas or the purge gas. The valves 14 include, for example, a first valve 141, a second valve 142, a third valve 143, a fourth valve 144, a fifth valve 145, and a sixth valve 146. The valve 14 may be configured by a valve electromagnetically driven, piezo driven, or motor driven. In the following description, the first valve 141, the second valve 142, the third valve 143, and the like are not distinguished from each other, and when they are described for the purpose of being collectively referred to, they are referred to as the valve 14.

The operations of the pump 13 and the valve 14 are controlled by a control unit 16 to be described later. The operation of only one of the pump 13 or the valve 14 may be controlled by the control unit 16 described later.

### Reservoir 15

The reservoir 15 includes, for example, a first reservoir 151 that stores sample gas and a second reservoir 152 that stores purge gas. The reservoir 15 may include only the first reservoir 151 that stores the sample gas.

Since the analysis device 1 includes the first reservoir 151, the sample gas collected through the first flow channel from the inside of the target toilet bowl 4A can be temporarily stored in the reservoir 133, and the sample gas to be supplied to the sensor unit 18 can be homogenized. Since the analysis device 1 supplies a certain amount of the sample gas stored in the first reservoir 151 to the sensor unit 18, the amount of the sample gas supplied to the sensor unit 18 becomes constant. As a result, the analysis accuracy of the sample gas can be further improved.

The first reservoir 151 is made of a material having flexibility in which an internal volume changes due to expansion, contraction, or deformation according to the amount of sample gas or purge gas stored therein. For example, the first reservoir 151 may be made of resin deformable according to the amount of gas stored therein, resin coated with metal, or the like.

Specifically, the first reservoir 151 is made of a material containing at least one of a vinyl alcohol based polymer, an ethylene-vinyl alcohol copolymer, polyethylene terephthalate, polyvinylidene fluoride, and a fluororesin.

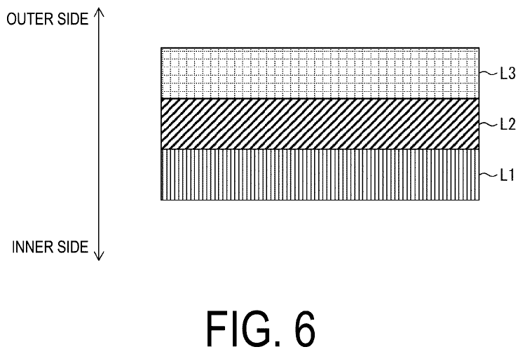

FIG. 6 is a diagram illustrating a configuration example of the first reservoir 151. As illustrated in FIG. 6, the first reservoir 151 may include a first layer L1 and a third layer L3 made of resin, and a second layer L2 made of metal. The first reservoir 151 may not have a three-layer structure as illustrated in FIG. 6. For example, the first reservoir 151 may have a two-layer structure including only the first layer L1 and the second layer L2. Alternatively, the first reservoir 151 may have a two-layer structure including only the second layer L1 and the third layer L2.

The first reservoir 151 may be subjected to a surface treatment for suppressing adhesion of the sample gas. In this case, the second layer L2 made of aluminum foil or steel foil is formed on the first layer L1 made of nylon, polyethylene, or the like.

When the first reservoir 151 does not include the second layer L2, surface treatment for reducing permeation of the sample gas may be performed. In this case, in the first reservoir 151, the third layer L3 is formed by aluminum vapor deposition, alumina vapor deposition, or silica vapor deposition. The third layer L3 may be made of polyvinylidene chloride.

When the first reservoir 151 has flexibility, it is possible to completely discharge the internal sample gas as compared with a reservoir having no flexibility. Therefore, the likelihood that the newly collected sample gas and the previously collected sample gas are mixed is reduced, and the analysis accuracy of the sample gas can be improved. Note that the first reservoir 151 is not limited to the above-described structure, and may have, for example, a five-layer structure in which a nylon layer, a polyethylene layer, an aluminum foil layer, a polyethylene layer, and a polyethylene layer are located in this order from the outer side toward the inner side.

The second reservoir 152 sucks air in a toilet room outside the toilet bowl 4A with a pump or the like, passes the air through a filter containing activated carbon or the like, and then stores the air as purge gas. Similarly to the first reservoir 151, the second reservoir 152 may be made of a material having flexibility whose internal volume changes by expansion, contraction, or deformation according to the amount of gas stored therein.

As described above, the analysis device 1 may not include the second reservoir 152. In this case, for example, the analysis device 1 may suck air in a toilet room outside the toilet bowl 4A as purge gas by a pump or the like and directly supply the purge gas to the first reservoir 151 or the sensor unit 18. The analysis device 1 may supply the purge gas to the first reservoir 151 or the sensor unit 18 from, for example, a cylinder in which the purge gas is stored in advance.

In the first reservoir 151, for example, an adsorbent that adsorbs gas other than the detection target contained in the sample gas such as silica gel or zeolite may be disposed. When the sample gas is concentrated in the first reservoir 151, for example, an adsorbent that adsorbs the gas to be detected contained in the sample gas, such as activated carbon and molecular sieve, may be disposed.

The first reservoir 151 may be provided with a heater for heating the sample gas. The heater for heating the sample gas may include, for example, a nichrome heater or a ceramic heater.

### Control unit 16

The control unit 16 includes an authentication unit 161, a gas analysis unit 162, a pump control unit 163, a valve control unit 164, a communication control unit 165, and an output control unit 166. The storage unit 17 stores user registration information 171. The storage unit 17 may be a storage unit in the analysis device 1 or an external storage communicably connected to the outside of the analysis device 1.

As described above, when the user sits on the toilet seat 4B, the authentication unit 161 acquires the weight information of the user detected by load sensor 111, and acquires user registration information 171 from storage unit 17 to authenticate the user. The authentication unit 161 acquires the name, the e-mail address, the user ID, or the like of the user from the extracted user information.

The gas analysis unit 162 analyzes the component contained in the sample gas. The gas analysis unit 162 detects and analyzes the type and concentration of gas contained in the sample gas based on the information sent from the sensor unit 18 described later. For example, the gas analysis unit 162 detects and analyzes the type and concentration of gas contained in the sample gas based on a voltage value or a current value sent from the sensor unit 18 described later. The gas analysis unit 162 may, for example, analyze the type and concentration of gas by AI.

The control unit 16 transmits the analysis result together with the user's name, e-mail address, user ID, or the like to the server device 2 via the communication control unit 165 described later. As described above, the server device 2 estimates the health condition of the user based on the transmitted analysis result, and transmits information indicating the estimated health condition to the terminal device 3.

The pump control unit 163 controls one or more pumps 13 included in the analysis device 1. The valve control unit 164 controls the plurality of valves 14 included in the analysis device 1.

As will be described in detail later, for example, the airflow generation unit 131 can be operated, or gas can be supplied to and discharged from the first reservoir 151, the second reservoir 152, and the sensor unit 18 through control of the pump 13 and the valve 14 by the pump control unit 163 and the valve control unit 164.

The control unit 16 includes the communication control unit 165 that controls the communication unit 19 and the output control unit 166 that controls the output unit 20.

### Storage Unit 17

The storage unit 17 stores various data and the like used by the control unit 16. As described above, the storage unit 17 stores the user registration information 171.

As described above, the user registration information 171 is, for example, a list in which weight information, fingerprint information, and user information are associated with each other. As an example, the user registration information 171 may be realized as a database that stores weight information, fingerprint information, and user information in association with each other. The user registration information 171 is stored in the storage unit 17 in advance.

### Sensor Unit 18

The sensor unit 18 is an analysis chamber including a plurality of sensors corresponding to a plurality of gases to be detected, and is formed with an internal space for accommodating the analysis target. The sample gas is supplied from the first reservoir 151 to the internal space of the sensor unit 18 by the second air pump 132, and the sensor unit 18 analyzes the supplied sample gas.

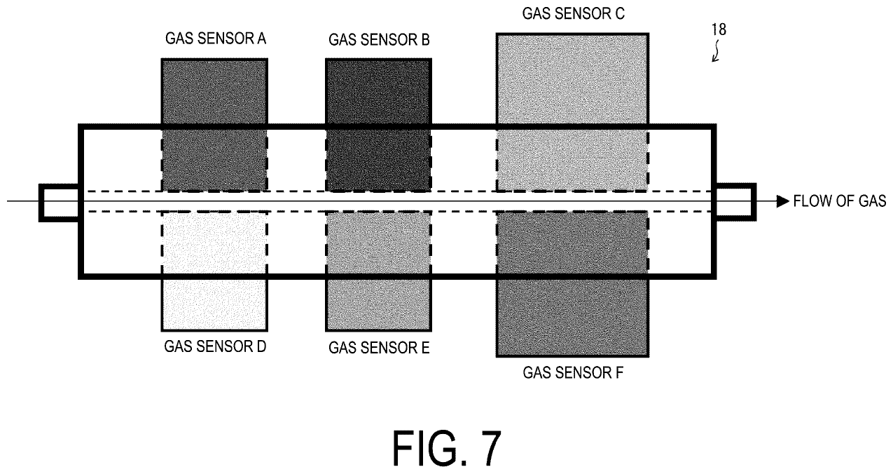

FIG. 7 is a diagram illustrating a configuration example of the sensor unit 18. As illustrated in FIG. 7, a flow channel through which gas such as sample gas and purge gas flows is provided inside the sensor unit 18. The volume of the flow channel is, that is, the volume of the internal space of the sensor unit 18. A plurality of gas sensors may be disposed so as to sandwich the flow channel. The sensor unit 18 illustrated in FIG. 7 includes gas sensors A to F. Detection surfaces capable of detecting gas components of the gas sensors A to F are exposed in the flow channel inside the sensor unit 18.

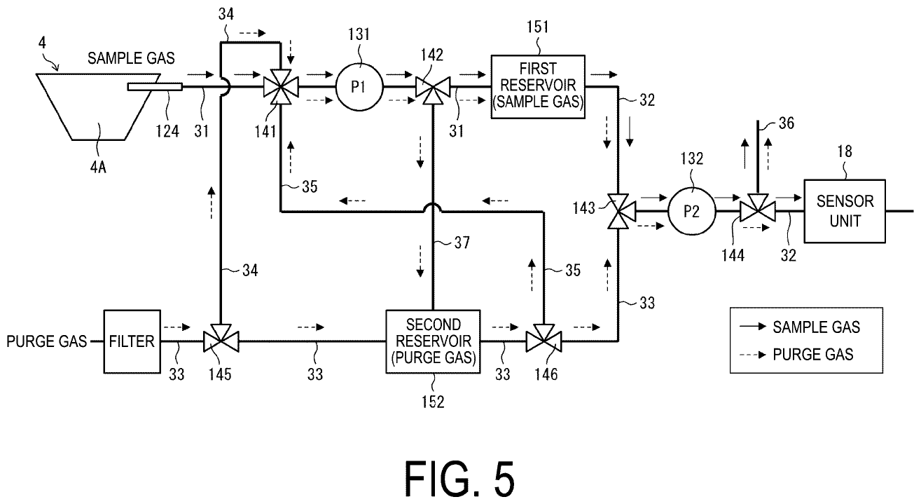

The volume of the internal space of the sensor unit 18 is smaller than the volume of the first reservoir 151, and the volume of the first reservoir 151 is greater than or equal to the sum of the volume of the sensor unit 18 and the internal volume of the second flow channel. Here, the internal volume of the second flow channel may be the sum of the volume of the internal space of the sensor unit 18, the internal volume of the second flow channel 32, the internal volume of the second air pump 132, and the internal volumes of the third valve 143 and the fourth valve 144 (see FIG. 5). As a result, an amount of sample gas suitable for the processing capacity of the sensor unit 18 is supplied into the sensor unit 18. For example, when the first reservoir 151 is formed of a material having flexibility, the “volume of the first reservoir 151” refers to the internal volume in a state where the first reservoir is most expanded.

The sensor unit 18 outputs a detection signal indicating a voltage value corresponding to the concentration of the specific gas contained in the sample gas to the gas analysis unit 162. For example, the sensor unit 18 outputs a detection signal indicating a voltage value corresponding to the concentration of the specific gas contained in the sample gas generated from feces to the gas analysis unit 162. For example, the sensor unit 18 may output a detection signal indicating a current value corresponding to the concentration of the specific gas contained in the sample gas generated from feces to the gas analysis unit 162.

The specific gas includes a specific gas to be detected and a specific gas other than the detection target. Examples of the specific gas to be detected include methane, hydrogen, carbon dioxide, methyl mercaptan, hydrogen sulfide, acetic acid, and trimethylamine. Examples of the specific gas other than the detection target includes ammonia and water.

Each of the plurality of sensors outputs a voltage corresponding to the concentration of at least any of these gases to the gas analysis unit 162. The sensor unit 18 may be a semiconductor sensor, a contact combustion sensor, an electrochemical sensor, a solid electrolyte sensor, or the like. For example, each of the plurality of sensors may output a current corresponding to the concentration of at least any of these gases to the gas analysis unit 162.

As will be described in detail later, the sensor unit 18 may output a detection signal to the gas analysis unit 162 every time a predetermined amount of sample gas and a predetermined amount of purge gas are alternately supplied to the sensor unit 18.

### Communication Unit 19 and Output Unit 20)

The analysis device 1 also includes a communication unit 19 for communicating with another device and an output unit 20 for outputting information. The communication unit 19 communicates with, for example, the server device 2 and the terminal device 3. The output unit 20 may be realized by a speaker, a display, or the like. The analysis device 1 may not include the output unit 20.

### Piping Diagram

FIG. 5 is a piping diagram of the analysis device 1 according to the first embodiment. As illustrated in FIG. 5, the sample gas wound up by the airflow generated by the airflow generation unit 12 is collected by the suction tube 124 disposed above the toilet bowl 4A.

### First Flow Channel 31

The first flow channel 31 is configured to include the suction tube 124, and connects a space in the toilet bowl 4A, from which the sample gas is to be collected, and the first reservoir 151. The first flow channel 31 is a flow channel for supplying sample gas collected from the inside of the toilet bowl 4A to the first reservoir 151 which is a reservoir. In the first flow channel 31, the first valve 141, the first air pump 131, the second valve 142, and the first reservoir 151 are arranged in this order from the upstream side.

The first air pump 131 supplies the sample gas in the toilet bowl 4A to the first reservoir 151 under the control of the pump control unit 163.

The first valve 141 is disposed between the suction tube 124 and the first air pump 131, and switches the connection state between the suction tube 124 and the first reservoir 151 under the control of the valve control unit 164. The first valve 141 also switches the connection state between the fourth flow channel 34 and the first air pump 131 and between the fifth flow channel 35 and the first air pump 131, to be described later.

The second valve 142 is disposed between the first air pump 131 and the first reservoir 151, and switches the connection state between the first air pump 131 and the first reservoir 151 under the control of the valve control unit 164.

### Second Flow Channel 32

The second flow channel 32 is a flow channel that connects the first reservoir 151 and the sensor unit 18 and supplies the sample gas in the first reservoir 151 to the sensor unit 18.

In the second flow channel 32, the first reservoir 151, the third valve 143, the second air pump 132, the fourth valve 144, and the sensor unit 18 are arranged in this order from the upstream side.

The third valve 143 is disposed between the first reservoir 151 and the second air pump 132, and switches the connection state between the first reservoir 151 and the second air pump 132 under the control of the valve control unit 164. The third valve 143 also switches the connection state between the third flow channel 33 to be described later and the second air pump 132.

The second air pump 132 supplies the sample gas or the purge gas in the first reservoir 151 to the sensor unit 18 under the control of the pump control unit 163.

The fourth valve 144 is disposed between the second air pump 132 and the sensor unit 18, and switches the connection state between the second air pump 132 and the sensor unit 18 under the control of the valve control unit 164. The fourth valve 144 also switches the connection state between the second air pump 132 and the toilet room, which is the outside. The fourth valve 144 and the outside are connected by a sixth flow channel 36.

As described above, the first reservoir 151 is made of a material that is deformable according to the amount of gas stored therein, and the first air pump 131 is disposed on the upstream and the second air pump 132 is disposed on the downstream of the first reservoir 151 with the first reservoir 151 interposed therebetween.

As will be described in detail later, after the second air pump 132 is operated to discharge the sample gas remaining in the first reservoir 151, the first air pump 131 is operated, and a new sample gas to be analyzed next can be supplied to the first reservoir 151.

As a result, it is possible to completely discharge the remaining sample gas as compared with a reservoir that has no flexibility, and thus it is possible to reduce the mixing of the newly collected sample gas and the remaining sample gas, and to improve the analysis accuracy of the sample gas.

### Third Flow Channel 33

The third flow channel 33 is a flow channel that connects the outside other than the toilet bowl 4A or the second reservoir 152 and the third valves 143, and supplies gas collected from the outside other than the inside of the toilet bowl 4A as purge gas.

In the third flow channel 33, a fifth valve 145, a second reservoir 152, a sixth valve 146, and a third valve 143 are arranged in this order from the upstream side. The third flow channel 33 may include a filter for removing a predetermined component from the purge gas on the upstream of the fifth valve 145. Here, the predetermined component may be, for example, a gas such as carbon dioxide, hydrogen, or methane, and/or an odor component such as methyl mercaptan or hydrogen sulfide.

The third flow channel 33 may not include the second reservoir 152. In this case, the third flow channel 33 does not include the sixth valve 146 and the fifth flow channel 35 to be described later together with the second reservoir 152. Here, a configuration not including the second reservoir 152 will be mainly described as a basis.

The fifth valve 145 is disposed at a branch between the third flow channel 33 and a fourth flow channel 34 to be described later, and switches the flow channel for guiding the purge gas between the third flow channel 33 and the fourth flow channel 34 under the control of the valve control unit 164.

The sixth valve 146 is disposed at a branch between the third flow channel 33 and a fifth flow channel 35 to be described later, and switches the flow channel for guiding the purge gas between the third flow channel 33 and the fifth flow channel 35 under the control of the valve control unit 164.

The third valve 143 is disposed on the most downstream side of the third flow channel 33. As described above, the third valve 143 switches the connection state between the third flow channel 33 and the second air pump 132.

The third flow channel 33 may be configured not to include the second reservoir 152. In this case, the third flow channel 33 may not include the sixth valve 146 and the fifth flow channel 35 to be described later together with the second reservoir 152.

### Fourth Flow Channel 34

The fourth flow channel 34 is a flow channel that connects the fifth valve 145 and the first valve 141 and supplies the first reservoir 151 with the purge gas collected from the outside other than the toilet bowl 4A. In the fourth flow channel 34, the fifth valve 145 and the first valve 141 are arranged in this order from the upstream side.

As described above, the fifth valve 145 is disposed at a branch between the third flow channel 33 and the fourth flow channel 34, and switches the flow channel for guiding the purge gas between the third flow channel 33 and the fourth flow channel 34 under the control of the valve control unit 164.

As described above, the first valve 141 switches the connection state between the fourth flow channel 34 and the first air pump 131.

### Fifth Flow Channel 35

The fifth flow channel 35 is a flow channel that connects the sixth valve 146 and the first valve 141 when the second reservoir 152 is provided, and supplies the purge gas stored in the second reservoir 152 to the first reservoir 151. In the fifth flow channel 35, the sixth valve 146 and the first valve 141 are arranged in this order from the upstream side.

As described above, the sixth valve 146 is disposed at a branch between the third flow channel 33 and the fifth flow channel 35, and switches the flow channel for guiding the purge gas between the third flow channel 33 and the fifth flow channel 35 under the control of the valve control unit 164.

As described above, the first valve 141 switches the connection state between the fifth flow channel 35 and the first air pump 131.

### Operation of Analysis System 100

### Cleaning of First Reservoir 151

The operation of the analysis device 1 according to the first embodiment will be described with reference to FIG. 5. As illustrated in FIG. 5, the control unit 16 may clean the first reservoir 151 before collecting the sample gas.

First, the control unit 16 discharges the previously collected sample gas remaining in the first reservoir 151. Specifically, the control unit 16 shuts off the second valve 142 located upstream of the first reservoir 151 and operates the second air pump 132 to discharge the sample gas remaining in the first reservoir 151 from the sixth flow channel 36 to the outside. At this time, the control unit 16 controls the third valve 143 and the fourth valve 144 so that the remaining sample gas can pass through the second flow channel 32 and the sixth flow channel 36.

Next, the control unit 16 shuts off the third valve 143 located downstream of the first reservoir 151 and operates the first air pump 131 to supply purge gas collected from the outside other than the toilet bowl 4A to the first reservoir 151 (supply of purge gas). At this time, the control unit 16 controls the fifth valve 145, the first valve 141, and the second valve 142 to allow the purge gas to pass through the third flow channel 33, the fourth flow channel 34, and the first flow channel 31. Note that the analysis device 1 may use gas collected from the inside of the toilet bowl 4A through a filter for removing a predetermined component as purge gas to be supplied to the first reservoir 151. Here, the predetermined component may be, for example, a gas such as carbon dioxide, hydrogen, or methane, and/or an odor component such as methyl mercaptan or hydrogen sulfide.

When the second reservoir 152 is provided, the control unit 16 may supply the purge gas stored in the second reservoir 152 to the first reservoir 151. In this case, the control unit 16 first supplies a purge gas collected from the outside other than the toilet bowl 4A to the second reservoir 152, and stores the purge gas in the second reservoir 152 in advance.

The seventh flow channel 37 is a flow channel that connects the second valve 142 and the second reservoir 152 and supplies a purge gas collected from the outside other than the toilet bowl 4A to the second reservoir 152. The control unit 16 shuts off the sixth valve 146 located downstream of the second reservoir 152 and operates the first air pump 131 to supply the purge gas collected from the outside other than the toilet bowl 4A to the second reservoir 152. At this time, the control unit 16 controls the fifth valve 145, the first valve 141, and the second valve 142 to allow the purge gas to pass through the third flow channel 33, the fourth flow channel 34, the first flow channel 31 (more specifically, the first flow channel 31 located between the first valve 141 and the second valve 142.), and the seventh flow channel 37.

Next, the control unit 16 supplies the purge gas stored in the second reservoir 152 to the first reservoir 151. At this time, the control unit 16 controls the sixth valve 146, the first valve 141, and the second valve 142 to allow the purge gas to pass through the third flow channel 33, the fifth flow channel 35, and the first flow channel 31.

Next, the control unit 16 shuts off the second valve 142 and operates the second air pump 132 to discharge the purge gas in the first reservoir 151 to the outside from the sixth flow channel 36 (discharge of the purge gas). At this time, the control unit 16 controls the third valve 143 and the fourth valve 144 to allow the purge gas to pass through the second flow channel 32 and the sixth flow channel 36.

The supply of the purge gas and the discharge of the purge gas to and from the first reservoir 151 described above may be performed over a plurality of times. As a result, the control unit 16 can more carefully clean the first reservoir 151 as compared with a case where the supply of the purge gas and the discharge of the purge gas are performed only once.

When cleaning the first reservoir 151, for example, the pump control unit 163 controls the first air pump 131 such that the flow rate of the first air pump 131 is greater than or equal to about 50 cm³ to less than or equal to about 10000 cm³ (for example, about 700 cm³) per minute. For example, the pump control unit 163 controls the second air pump 132 such that the flow rate of the second air pump 132 is greater than or equal to about 1 cm³ and less than or equal to about 700 cm³ (for example, about 100 cm³) per minute.

Here, the flow rate of the second air pump 132 when supplying the purge gas is controlled to be a flow rate higher than the flow rate (about 50 cm³ per minute) of when supplying the sample gas or the purge gas to the sensor unit 18, which will be described in detail later. This is because the discharge of the purge gas to the outside by the second air pump 132 does not need to take into consideration the processing capacity of the sensor unit 18, unlike the supply of the sample gas or the purge gas to the sensor unit 18. Accordingly, since the second air pump 132 can discharge the entire amount of the purge gas to the outside, the control unit 16 can quickly clean the first reservoir 151.

### Storage of Sample Gas

Next, the control unit 16 collects the sample gas and stores the sample gas in the first reservoir 151. The control unit 16 shuts off the third valve 143 located downstream of the first reservoir 151 and operates the first air pump 131 to newly collect sample gas from the toilet bowl 4A and supply the sample gas to the first reservoir 151. At this time, the control unit 16 controls the first valve 141 and the second valve 142 to allow the sample gas to pass through the first flow channel 31.

For example, the pump control unit 163 controls the first air pump 131 such that the flow rate of the first air pump 131 is greater than or equal to about 50 cm³ and less than or equal to about 10000 cm³ (for example, about 700 cm³) per minute. For example, the maximum flow rate of the first air pump 131 is about 10000 cm³ per minute, the average flow rate is about 700 cm³ per minute, and the minimum flow rate is about 50 cm³ per minute. Instead of the minimum flow rate, for example, an instantaneous flow rate or a momentary flow rate may be used. In this case, the instantaneous flow rate or the momentary flow rate may be about 1 cm³ per minute.

### Measurement of Purge Gas

Next, the control unit 16 detects a component contained in the purge gas by the sensor unit 18. Here, in order to exclude the influence of the purge gas from the sample gas detection result, the control unit 16 may execute the measurement of the purge gas before the measurement of the sample gas. Alternatively, by alternately measuring the sample gas and the purge gas, the control unit 16 may acquire data related to switching of waveform data between the time of sample gas measurement and the time of purge gas measurement. For example, when the sample gas is switched to the purge gas, the waveform (ON waveform) for the time of sample gas measurement is switched to the waveform (OFF waveform) for the time of purge gas measurement. Therefore, in addition to the saturation value of the waveform, a prediction model created based on machine learning using data (i.e., data related to inclination, rising, and falling is included.) indicating a waveform pattern observed at the time of switching of the ON waveform/OFF waveform as an explanatory variable may be applied to the control unit 16. By applying such a prediction model, the control unit 16 can improve the detection accuracy of the component contained in the sample gas.

When the second reservoir 152 is not provided, the control unit 16 may supply purge gas collected from the outside other than the toilet bowl 4A to the sensor unit 18. For example, the control unit 16 may operate the second air pump 132 to control the fifth valve 145, the third valve 143, and the fourth valve 144 so that the purge gas can pass through the third flow channel 33 and the second flow channel 32.

When the second reservoir 152 is provided, the control unit 16 may supply the purge gas stored in the second reservoir 152 to the sensor unit 18. In this case, the control unit 16 first supplies a purge gas collected from the outside other than the toilet bowl 4A to the second reservoir 152, and stores the purge gas in the second reservoir 152 in advance. As described above, the control unit 16 supplies the purge gas collected from the outside other than the toilet bowl 4A to the second reservoir 152 by shutting off the sixth valve 146 located downstream of the second reservoir 152 and operating the first air pump 131. At this time, the control unit 16 controls the fifth valve 145, the first valve 141, and the second valve 142 to allow the purge gas to pass through the third flow channel 33, the fourth flow channel 34, the first flow channel 31 (more specifically, the first flow channel 31 located between the first valve 141 and the second valve 142.), and the seventh flow channel 37. Note that the control unit 16 may use gas collected from the inside of the toilet bowl 4A through a filter for removing a predetermined component as purge gas to be supplied to the second reservoir 152. Here, the predetermined component may be, for example, a gas such as carbon dioxide, hydrogen, or methane, and/or an odor component such as methyl mercaptan or hydrogen sulfide. By using this filter, the control unit 16 can even store the gas in the toilet bowl 4A in the second reservoir 152 and use the gas as the purge gas.

Next, the control unit 16 may shut off the fifth valve 145 located upstream of the second reservoir 152 and operate the second air pump 132 to supply the purge gas from the second reservoir 152 to the sensor unit 18. At this time, the control unit 16 controls the sixth valve 146, the third valve 143, and the fourth valve 144 to allow the purge gas to pass through the third flow channel 33 and second flow channel 32.

At this time, for example, the pump control unit 163 controls the second air pump 132 such that the flow rate of the second air pump 132 is greater than or equal to about 1 cm³ and less than or equal to about 700 cm³ (for example, about 50 cm³) per minute. This is a smaller flow rate than when a flow rate at which the first air pump 131 stores the sample gas in the first reservoir 151 is about 700 cm³ per minute. For example, the maximum flow rate of the second air pump 132 is about 700 cm³ per minute, the average flow rate is about 50 cm³ per minute, and the minimum flow rate is about 1 cm³ per minute. Instead of the minimum flow rate, for example, an instantaneous flow rate or a momentary flow rate may be used. In this case, the instantaneous flow rate or the momentary flow rate may be about 1 cm³ per minute.

The pump control unit 163 controls the purge gas supply time corresponding to the response speed of the sensor unit 18 so as not to exceed the processing capacity of the sensor unit 18. Therefore, the flow rate of the second air pump 132 is smaller than that of the first air pump 132 in order for the second air pump 131 to correspond to the response speed of the sensor unit 18.

As a result, the control unit 16 can supply the purge gas to the sensor unit 18 at an appropriate flow rate that does not exceed the processing capacity of the sensor unit 18, and can quickly store the sample gas in the first reservoir 151. Thus, the time required for the user to sit on the toilet seat can be shortened.

### Measurement of Sample Gas

Next, the control unit 16 causes the sensor unit 18 to detect a component contained in the sample gas stored in the first reservoir 151. The control unit 16 shuts off the second valve 142 located upstream of the first reservoir 151 and operates the second air pump 132 to supply the sample gas in the first reservoir 151 to the sensor unit 18. At this time, the control unit 16 controls the third valve 143 and the fourth valve 144 to allow the sample gas to pass through the second flow channel 32. As a result, the sensor unit 18 can detect the component contained in the sample gas.

At this time, the pump control unit 163 controls the second air pump 132 such that the flow rate of the second air pump 132 is greater than or equal to about 1 cm³ and less than or equal to about 700 cm³ (for example, about 50 cm³) per minute which is less than the flow rate of the first air pump 131. The flow rate of the second air pump 132 is smaller than when the flow rate At which the first air pump 131 stores the sample gas in the first reservoir 151 is about 700 cm³ per minute.

This is because the pump control unit 163 controls the flow rate of the sample gas to supply to the sensor unit 18 so as to be an appropriate flow rate that does not exceed the processing capacity of the sensor unit 18. As a result, the flow rate of the second air pump 132 becomes smaller than that of the first air pump 131.

Thus, the control unit 16 can supply the sample gas to the sensor unit 18 at an appropriate flow rate that does not exceed the processing capacity of the sensor unit 18.

### Alternate Measurement

Next, the control unit 16 detects the component contained in the purge gas again by the sensor unit 18. The control unit 16 operates the second air pump 132 to supply the purge gas collected from the outside other than the toilet bowl 4A to the sensor unit 18. Thus, the sensor unit 18 can detect the component contained in the purge gas.

The control unit 16 controls the third valve 143 to alternately supply the sample gas and the purge gas to the sensor unit 18 over a plurality of times. Each time the sample gas and the purge gas are alternately supplied, the sensor unit 18 detects the component contained in the sample gas and the purge gas. The number of times of alternately supplying is not particularly limited, but may be, for example, about three times. When a plurality of sample gases are detected, the sensor unit 18 may select second and subsequent waveform data and estimate a component contained in the sample gas and its concentration based on the waveform data. The control unit 16 can improve the accuracy of gas concentration estimation by using a prediction model that performs machine learning with the waveform data at the time of sample gas detection and the waveform data at the time of purge gas detection as explanatory variables. For this machine learning, for example, a linear regression method or a neural network can be used.

### Discharge of Sample Gas

Next, the control unit 16 discharges the sample gas remaining in the first reservoir 151. The control unit 16 shuts off the second valve 142 located upstream of the first reservoir 151 and operates the second air pump 132 to discharge the sample gas remaining in the first reservoir 151 from the sixth flow channel 36 to the outside. At this time, the control unit 16 controls the third valve 143 and the fourth valve 144 so that the remaining sample gas can pass through the second flow channel 32 and the sixth flow channel 36.

### Flow of Processes by Control Unit

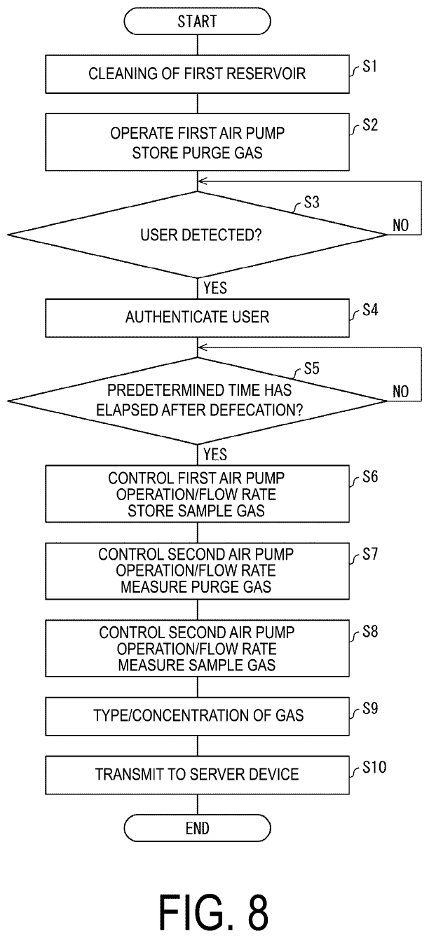

FIG. 8 is a flowchart illustrating an example of a flow of processes executed by the control unit 16 illustrated in FIG. 3. A flow of processes executed by the control unit 16 will be described with reference to FIG. 8.

As illustrated in FIG. 8, the control unit 16 cleans the first reservoir 151 before collecting the sample gas (S1). First, the control unit 16 discharges the previously collected sample gas remaining in the first reservoir 151. Then, after supplying the purge gas to the first reservoir 151, the control unit 16 discharges the purge gas in the first reservoir 151 to the outside. Such supply and discharge of the purge gas may be performed a plurality of times.

Next, the pump control unit 163 operates the first air pump 131 to store the purge gas collected from the outside other than the toilet bowl 4A in the second reservoir 152 (S2).

The user detection unit 11 determines whether or not a user is detected (S3). When the user detection unit 11 detects the user (YES in S3), the authentication unit 161 authenticates the user based on the information acquired by the load sensor 111 and the fingerprint sensor 112, and the user registration information 171 (S4).

On the other hand, when the user detection unit 11 does not detect a user (NO in the S3), the process returns to the process of S3.

The control unit 16 determines whether or not a predetermined time has elapsed after defecation of the user (S5). The predetermined time may be, for example, 90 seconds later, and is not particularly limited.

When the control unit 16 determines that the predetermined time has elapsed after the defecation of the user (YES in S5), the pump control unit 163 operates the first air pump 131 to store the sample gas collected from the suction tube 124 in the first reservoir 151 (S6). At this time, the pump control unit 163 controls the flow rate of the first air pump 131 to be, for example, greater than or equal to about 50 cm³ and less than or equal to about 10000 cm³ (for example, about 700 cm³) per minute.

On the other hand, when the control unit 16 determines that the predetermined time has not elapsed after the defecation of the user (NO in S5), the process returns to the process of S5.

Next, the pump control unit 163 operates the second air pump 132 to supply the purge gas collected from the outside other than the toilet bowl 4A to the sensor unit 18 (S7). At this time, the pump control unit 163 controls the flow rate of the second air pump 132 to be smaller than the flow rate of the first air pump 131, for example, to be greater than or equal to about 1 cm³ and less than or equal to about 700 cm³ per minute (for example, about 50 cm³).

As a result, the control unit 16 can supply the purge gas to the sensor unit 18 at an appropriate flow rate that does not exceed the processing capacity of the sensor unit 18, and can quickly store the sample gas in the first reservoir 151.

Next, the pump control unit 163 operates the second air pump 132 to supply the sample gas stored in the first reservoir 151 to the sensor unit 18 (S8). At this time, the pump control unit 163 controls the flow rate of the second air pump 132 to be smaller than the flow rate of the first air pump 131, for example, to be greater than or equal to about 1 cm³ and less than or equal to about 700 cm³ per minute (for example, about 50 cm³).

As a result, the control unit 16 can supply the sample gas to the sensor unit 18 at an appropriate flow rate that does not exceed the processing capacity of the sensor unit 18, and can quickly store the sample gas in the first reservoir 151.

The control unit 16 alternately repeats the process of S7 process and the process of S8 over a plurality of times.

The sensor unit 18 detects a component contained in the sample gas or the purge gas and sends the component to the gas analysis unit 162 every time the sample gas and the purge gas are alternately supplied. The gas analysis unit 162 analyzes the type and concentration of gas based on the sent data (S9).

The control unit 16 transmits data on the type/concentration of the analyzed gas to the server device 2 via the communication unit 19 (S10), and ends the process.

The server device 2 estimates the health condition of the user based on the transmitted data. The server device 2 transmits data related to the health condition of the user to the terminal device 3 of the user.

### Second Embodiment

A second embodiment of the present disclosure will be described below. For the sake of convenience of description, components having the same functions as those described in the first embodiment are denoted by the same reference numerals, and the description thereof will not be repeated. The same applies to the second and subsequent embodiments.

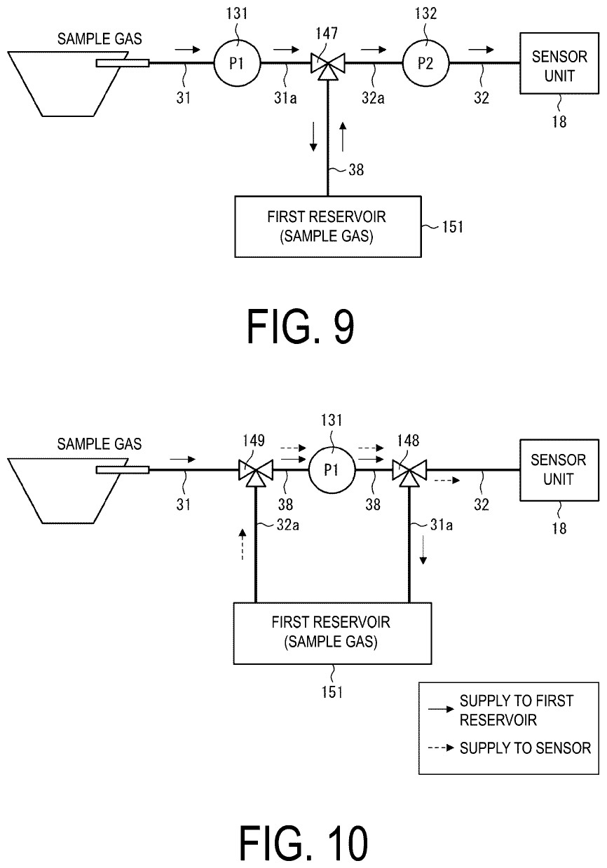

FIG. 9 is a part of a piping diagram of the analysis device 1 according to a second embodiment. As illustrated in FIG. 9, the analysis device 1 according to the second embodiment is common to the first embodiment in that the analysis device includes a first reservoir 151 having flexibility, a first air pump 131, a second air pump 132, a sensor unit 18, a first flow channel 31, and a second flow channel 32. However, the analysis device 1 according to the second embodiment is different from the first embodiment in that the first air pump 131, the second air pump 132, and the first reservoir 151 are arranged in a T-shape.

The first flow channel 31 and the second flow channel 32 include a common flow channel 38 that is common to each other and dedicated flow channels 31a and 32a that are not common to each other. Specifically, the common flow channel 38 functioning as the first flow channel 31 and the second flow channel, the dedicated flow channel 31a functioning only as the first flow channel 31, and the dedicated flow channel 32a functioning only as the second flow channel 32 are provided.

The dedicated flow channel 31a, the dedicated flow channel 32a, and the common flow channel 38 are connected to each other so as to form a T-shape, and the seventh valve 147 is disposed at connecting portions of the three flow channels.

The first air pump 131 is disposed in the dedicated flow channel 31a, and the second air pump 132 is disposed in the dedicated flow channel 32a. The first air pump 131 may interiorly include a dedicated flow channel 31a and may configure at least a part of the dedicated flow channel 31a. The second air pump 132 may interiorly include a dedicated flow channel 32a and may configure at least a part of the dedicated flow channel 32a. In this case, the seventh valve 147 is directly connected to the discharge port of the first air pump 131 and the inflow port of the second air pump 132.

When one of the first air pump 131 and the second air pump 132 is operating, the other is stopped. Specifically, by stopping the second air pump 132 and operating the first air pump 131, the control unit 16 moves the sample gas along the dedicated flow channel 31a and the common flow channel 38, and supplies the sample gas to the first reservoir 151. At this time, the valve control unit 164 controls the seventh valve 147 to shut off the connection between the dedicated flow channel 32a and the common flow channel 38 and release the connection between the dedicated flow channel 31a and the common flow channel 38.

By stopping the first air pump 131 and operating the second air pump 132, the control unit 16 moves the sample gas in the first reservoir 151 along the common flow channel 38 and the dedicated flow channel 32a, and supplies the sample gas to the sensor unit 18. At this time, the valve control unit 164 controls the seventh valve 147 to shut off the connection between the common flow channel 38 and the dedicated flow channel 31a and release the connection between the common flow channel 38 and the dedicated flow channel 32a.

The flow rate of the first air pump 131 is about 700 cm³ per minute, and the flow rate of the second air pump 132 is about 50 cm³ per minute. As a result, the control unit 16 can supply the sample gas to the sensor unit 18 at an appropriate flow rate that does not exceed the processing capacity of the sensor unit 18, and can quickly store the sample gas in the first reservoir 151.

### Third Embodiment

FIG. 10 is a part of a piping diagram of the analysis device 1 according to a third embodiment. As illustrated in FIG. 10, the analysis device 1 according to the third embodiment includes the first air pump 131, but is different from the first embodiment and the second embodiment in that it does not include the second air pump 132. The first air pump 131 also functions as the second air pump 132.

The analysis device 1 includes a first reservoir 151 having flexibility, a first air pump 131, a sensor unit 18, a first flow channel 31, and a second flow channel 32. The first flow channel 31 and the second flow channel 32 include a common flow channel 38 and dedicated flow channels 31a and 32a.

An eighth valve 148 is disposed at the connecting portion between the common flow channel 38 and the dedicated flow channel 31a, and a ninth valve 149 is disposed at the connecting portion between the common flow channel 38 and the dedicated flow channel 32a.

The first air pump 131 is disposed in the common flow channel 38 between the eighth valve 148 and the ninth valve 149. The first air pump 131 may interiorly include a common flow channel 38 and constitute at least a part of the common flow channel 38. The flow channel included inside the first air pump 131 can function as at least a part of the common flow channel 38. For example, the eighth valve 148 may be directly connected to the discharge port of the first air pump 131, and the ninth valve 149 may be directly connected to the inflow port of the first air pump 131.

The valve control unit 164 controls the eighth valve 148 and the ninth valve 149 to release the connection between the first flow channel 31 and the common flow channel 38 and to release the connection between the common flow channel 38 and the dedicated flow channel 31a. When the first air pump 131 is operated in this state, the sample gas is supplied to the first reservoir 151 through the common flow channel 38 and the dedicated flow channel 31a.

The valve control unit 164 controls the eighth valve 148 and the ninth valve 149 to release the connection between the dedicated flow channel 32a and the common flow channel 38 and to release the connection between the common flow channel 38 and the second flow channel 32. When the first air pump 131 is operated in this state, the sample gas in the first reservoir 151 is passed through the dedicated flow channel 32a, the common flow channel 38, and the second flow channel 32, and is supplied to the sensor unit 18.

The flow rate when the first air pump 131 supplies the sample gas to the first reservoir 151 is about 700 cm³ per minute, and the flow rate when the first air pump 131 supplies the sample gas to the sensor unit 18 is controlled by the pump control unit 163 so as to be about 50 cm³ per minute.

As a result, the control unit 16 can quickly store the sample gas in the first reservoir 151 while supplying the sample gas to the sensor unit 18 at an appropriate flow rate that does not exceed the processing capacity of the sensor unit 18.

### Fourth Embodiment

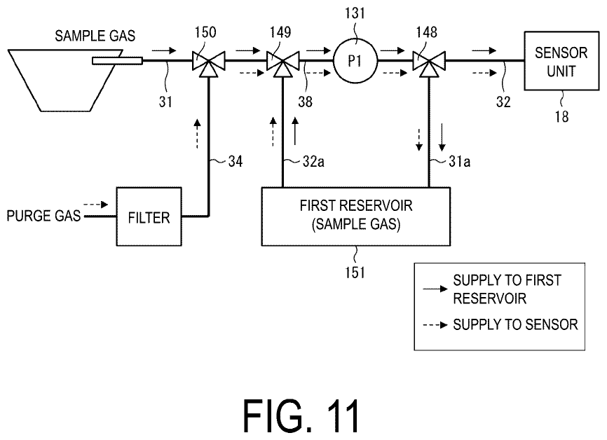

FIG. 11 is a part of a piping diagram of the analysis device 1 according to a fourth embodiment. As illustrated in FIG. 9, the analysis device 1 according to the fourth embodiment is different from the third embodiment in that the second reservoir 152 for storing the purge gas is not provided, and the purge gas collected from the outside other than the toilet bowl 4A is supplied as it is without being stored. In FIG. 10, illustration of the second reservoir 152 is omitted.

The analysis device 1 further includes a fourth flow channel 34 for supplying the purge gas, a filter for filtering the purge gas, and a tenth valve 150. The tenth valve 150 is disposed at a connecting portion between the first flow channel 31 and the fourth flow channel 34, and switches the gas to be supplied to the first reservoir 151 or the sensor unit 18 between the sample gas and the purge gas. This eliminates the need for the analysis device 1 to include the second reservoir 152 and the second air pump 132, making it possible to make the analysis device 1 compact.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

### REFERENCE SIGNS

1 Analysis device
4A Toilet bowl
18 Sensor unit (analysis chamber)
31 First flow channel
31a Dedicated flow channel
32 Second flow channel
32a Dedicated flow channel
38 Common flow channel
131 First air pump (first pump)
132 Second air pump (second pump)
151 First reservoir (reservoir)
161 Authentication unit

1. An analysis device comprising:
a reservoir that stores sample gas collected through a first flow channel;
an analysis chamber for analyzing the sample gas supplied from the reservoir; and
a second flow channel connecting the reservoir and the analysis chamber; wherein
a volume of the analysis chamber is smaller than a volume of the reservoir, and
a volume of the reservoir is greater than or equal to a sum of a volume of the analysis chamber and a volume of the second flow channel.

2. The analysis device according to claim 1, wherein
the reservoir is made of a material having flexibility.

3. The analysis device according to claim 1, further comprising:
one or more pumps capable of moving sample gas along at least one of the first flow channel and the second flow channel; wherein
a flow rate of the sample gas in the first flow channel is greater than or equal to a flow rate of the sample gas in the second flow channel.

4. The analysis device according to claim 3, wherein
the first flow channel and the second flow channel have a common flow channel that is shared by the first flow channel and the second flow channel and a dedicated flow channel that is not shared by the first flow channel and the second flow channel, and the pump constitutes at least a part of the common flow channel.

5. The analysis device according to claim 3, wherein
the first flow channel and the second flow channel have a common flow channel that is shared by the first flow channel and the second flow channel and a dedicated flow channel that is not shared by the first flow channel and the second flow channel, and the pump comprises a first pump that constitutes at least a part of the dedicated flow channel of the first flow channel and a second pump that constitutes at least a part of the dedicated flow channel of the second flow channel.

6. The analysis device according to claim 5, wherein
when one of the first pump and the second pump is operating, the other is stopped.

7. The analysis device according to claim 1, wherein
a material forming the reservoir comprises at least one selected from the group consisting of a vinyl alcohol-based polymer, an ethylene-vinyl alcohol copolymer, polyethylene terephthalate, polyvinylidene fluoride, and fluororesin.

8. The analysis device according to claim 7, wherein
the reservoir is formed by performing a surface treatment for suppressing adhesion of the sample gas or a surface treatment for reducing transmission of the sample gas on a film surface made of the material or a metal foil located on the film surface.

9. An analysis device according to claim 1, wherein
a flow rate of the sample gas in the first flow channel when the sample gas is fed into the reservoir is greater than or equal to 50 cm³ and less than or equal to 10000 cm³ per minute; and
a flow rate of the sample gas in the second flow channel when the stored sample gas is sent from the reservoir to the analysis chamber is greater than or equal to 1 cm³ and less than or equal to 700 cm³ per minute.

10. The analysis device according to claim 1, wherein
the sample gas is a gas collected from a space in a toilet bowl,
an authentication unit that authenticates a user who uses the toilet bowl is further provided, and
the sample gas is stored in the reservoir after the user is authenticated by the authentication unit.

11. The analysis device according to claim 10, wherein
the sample gas is stored in the reservoir after a predetermined time has elapsed since the user is authenticated by the authentication unit.

12. The analysis device according to claim 1, wherein
the sample gas is a gas collected from a space in a toilet bowl,
an authentication unit that authenticates a user who uses the toilet bowl is further provided, and
the authentication unit authenticates the user after sample gas is stored in the reservoir.

13. The analysis device according to claim 10, wherein
the authentication unit authenticates the user based on a physical feature of the user.

14. The analysis device according to claim 13, wherein
the physical feature is a fingerprint and/or a weight of the user.

15. The analysis device according to claim 1, further comprising:
a sensor for outputting a voltage or a current corresponding to a concentration of a specific gas contained in the sample gas.

16. The analysis device according to claim 1, further comprising:
a first pump located in the first flow channel; and
a second pump located in the second flow channel.

17. The analysis device according to claim 1, further comprising a pump for supplying a sample gas and a purge gas to the reservoir and the analysis chamber, wherein
a flow rate of a gas supplied by the pump when the reservoir is cleaned is greater than a flow rate of a gas supplied by the pump when the analysis chamber analyzes the sample gas.

18. The analysis device according to claim 5, wherein a flow rate of the first pump is greater than a flow rate of the second pump.
